(19) United States
(12) Patent Application Publication
(10) Pub. No.: US 2013/0103161 A1
(43) Pub. Date: Apr. 25, 2013

(54) Iron Based Alloys for Bioabsorbable Stent

(71) Applicant: Medtronic Vascular, Inc., Santa Rosa, CA (US)

(72) Inventors: Ya Guo, Santa Rosa, CA (US); Joseph Berglund, Santa Rosa, CA (US); Christopher W. Storment, Sonoma, CA (US); James W. Wright, Wilmette, IL (US)

(73) Assignee: MEDTRONIC VASCULAR, INC., Santa Rosa, CA (US)

(21) Appl. No.: 13/656,163

(22) Filed: Oct. 19, 2012

Related U.S. Application Data
(60) Provisional application No. 61/549,712, filed on Oct. 20, 2011.

(51) Int. Cl. A61F 2/82

(52) U.S. Cl. 623/23.7

(57) ABSTRACT

A stent includes an iron-based alloy that consists essentially of: Fe—X—Y, wherein X is at least one austenite stabilizing element selected from the group consisting of Co, Ni, Mn, Cu, Re, Rh, Ru, Ir, Pt, Pd, C, and N, and wherein Y is at least one corrosion-activator species selected from the group consisting of Au, and Pd.

### CROSS-REFERENCE TO RELATED APPLICATIONS

This application claims the benefit of priority from U.S. Provisional Patent Application No. 61/549,712, filed Oct. 20, 2011, the entire content of which is incorporated herein by reference.

### FIELD

The present invention is related to iron based alloys for a bioabsorbable stent.

### BACKGROUND

Bioabsorbable metals that degrade via corrosion are attractive material candidates for bioabsorbable stents, because bioabsorbable metals are inherently stiffer and stronger than the typical polymeric materials that have been previously considered for such applications. Of the metals that have been previously evaluated, magnesium (Mg) has typically been selected as the primary alloying element due to its good biocompatibility properties. Two challenges with predominantly magnesium based alloys are 1) their hexagonal close-packed (“HCP”) crystal structure impedes ductility and may lead to brittle fracture and 2) they may corrode too quickly, thereby allowing the device to lose its structure before adequate healing, endothelialization, and incorporation into the surrounding tissue has occurred.

Iron (Fe) based alloys demonstrate superior strength, ductility and corrosion resistance compared to other metallic materials such as magnesium (Mg) based alloys. Additionally, the microstructure of Fe-based system may be tailored using controlled heat treatment to produce a wide range of phases that include austenite and martensite phases. Fe-based alloys are possible alternatives for biodegradable medical implant applications, such as stents.

One of the challenges of iron is low degradation rate in biological environment compared to Mg-based alloys due to the formation of a passivating iron oxide layer on the surface. One strategy to increase the corrosion rate of Fe-based materials is through the precipitation of cathodic second-phase particles; however, according to Mueller et al., US 2009/0198320 A1, this may result in crevice corrosion and pitting corrosion. Because implantable devices are envisioned to have potentially thin cross-sections, there is a need for an iron-based alloy with microscopically uniform corrosion at rates higher than pure iron. For example, if the two-phase alloy has palladium (Pd)-rich second phases that are about 0.5 to 5 microns in diameter, and a structural component of the stent has a diameter of 50 microns, the pitting corrosion sites may constitute greater than 10% the cross-sectional thickness, which may lead to rapid fracture of the structural component of the stent.

### SUMMARY

In accordance with an aspect of embodiments of the present invention, there is provided a stent comprising an iron-based alloy consisting essentially of: Fe—X—Y, wherein X is at least one austenite stabilizing element selected from the group consisting of Co, Ni, Mn, Cu, Re, Rh, Ru, Ir, Pt, Pd, C, and N, and wherein Y is at least one corrosion-activator species selected from the group consisting of Au, and Pd.

In an embodiment, the stent comprises an alloy consisting essentially of 64 weight % Fe, 35 weight % Mn, and 1 weight % Au.

In an embodiment, the stent comprises an alloy consisting essentially of 64.5 weight % Fe, 35 weight % Mn, and 0.5 weight % Pd.

In an embodiment, the stent comprises an alloy consisting essentially of 70 weight % Fe, and 30 weight % Pd.

In an embodiment, the stent comprises an alloy consisting essentially of 63 weight % Fe, 35 weight % Pt, and 2 weight % Au.

In an embodiment, the stent comprises an alloy consisting essentially of 60 weight % Fe, 35 weight % Pt, and 5 weight % Pd.

In an embodiment, the stent comprises an alloy consisting essentially of 63 weight % Fe, 35 weight % Ir, and 2 weight % Au.

In an embodiment, the stent comprises an alloy consisting essentially of 60 weight % Fe, 35 weight % Ir, and 5 weight % Pd.

In an embodiment, the stent comprises an alloy consisting essentially of 54.8 weight % Fe, 45 weight % Mn, and 0.18 weight % Au.

In an embodiment, the stent comprises an alloy consisting essentially of 55.8 weight % Fe, 44 weight % Mn, and 0.18 weight % Au.

In an embodiment, the stent further comprises less than 1% phase fraction of a second-phase particle configured to limit grain growth during processing of the alloy.

In an embodiment, the second-phase particle is selected from the group consisting of: NbC, TiC, VC, and VN.

In an embodiment, the stent further comprises a plurality of struts and a plurality of turns, wherein each turn connects a pair of adjacent struts.

### BRIEF DESCRIPTION OF THE DRAWINGS

FIG. 1 illustrates a stent in accordance with embodiments of the present invention; and

FIG. 2 illustrates a periodic table of elements showing the surface energy (top number) and Pauling electronegativity (bottom) for each element relative to Fe.

### DETAILED DESCRIPTION

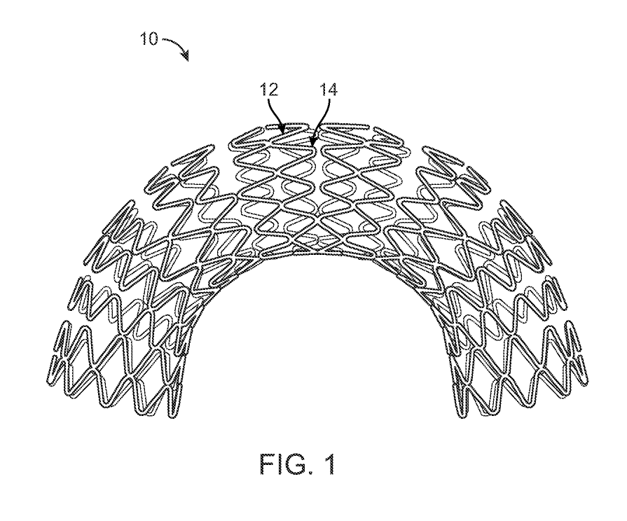

FIG. 1 illustrates a stent 10 that includes a plurality of struts 12 and a plurality of crowns or turns 14, with each crown or turn 14 connecting a pair of adjacent struts 12. The stent 10 may be formed from a tube by methods known in the art, such as laser cutting. The tube used to form the stent 10 may be made in accordance with embodiments of the present invention disclosed herein. The stent 10 may also be formed from at least one elongate member such as, for example, a wire. The stent 10 may be formed from a single wire shaped into a continuous sinusoid that is wrapped to form a stent framework.

Fe-based materials containing the body-centered cubic (“BCC”) martensite phase are ferromagnetic and less suitable for implant applications due to challenges associated with testing, such as magnetic resonance testing (“MRI”) scans. Therefore, it is desirable to ensure the matrix phases are paramagnetic to be suitable for medical applications. Alloys in accordance with embodiments of the invention are predominantly face-centered cubic (“FCC”) austenite with only HCP (epsilon) as transformation products during cold-work, both of which are not ferromagnetic and not expected to interfere with MRI. The elements selected for the function of stabilizing the FCC phase or activating corrosion are constrained to be biologically compatible.

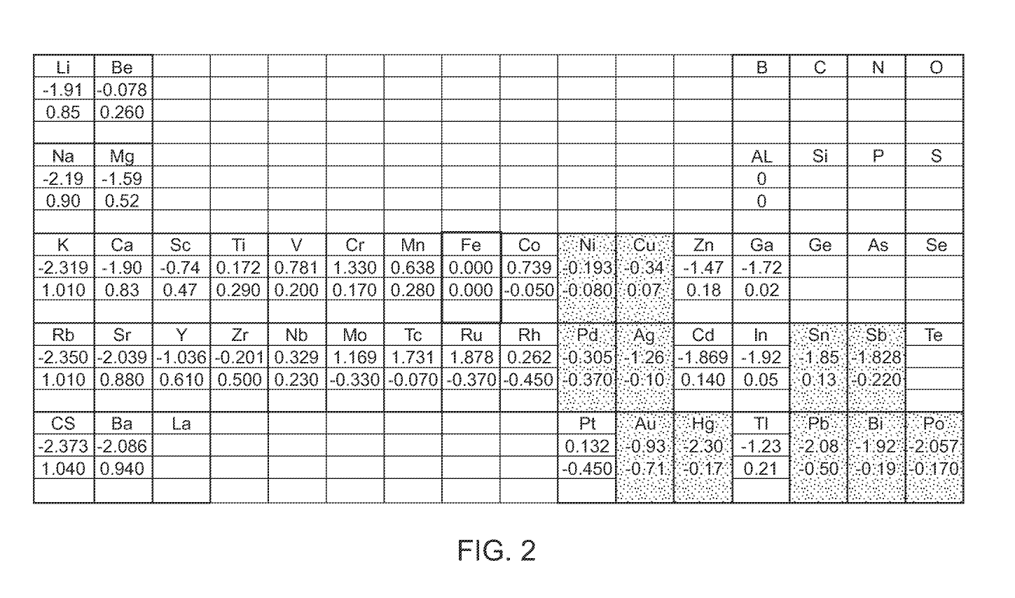

A corrosion activation model that can predict the effect of alloying elements on the corrosion properties like open circuit potential (OCP) has been developed. Specifically, the model describes which alloying elements will activate the host material and the level to which the alloying elements will activate the host material. The activation model assumes that activator elements segregate to the surface of the host material and at which point the activator elements facilitate corrosion by “destabilizing” the surface. The surface segregation portion is captured by surface energies: alloying elements with a lower surface energy will segregate to the surface. The “destabilization” aspect is captured with Pauling electronegativity: alloying elements with a higher electronegitivity will attract electron density towards itself making it easier to oxidize (or corrode) the host material. FIG. 2 illustrates a periodic table of elements showing the surface energy (top number) and Pauling electronegativity (bottom) for each element relative to Fe. The shaded elements in FIG. 2 pass both criteria for corrosion activation of iron. It can be seen that the two preferred non-toxic and Fe-soluble activator species for Fe are Pd and Au, as shown in FIG. 2. The model predicts that the onset of corrosion activation will occur at 0.006 atomic % gold and will reach a saturation at about 0.09 atomic % gold, beyond which corrosion rate does not increase with further dissolved gold.

To stabilize the FCC austenite phase, numerous elements can be utilized alone or in combination. Secondary considerations for the element selection include bio-compatibility and radiopacity. The list of potential alloying elements to stabilize austenite in accordance with embodiments of the present invention include: Co, Ni, Mn, Cu, Re, Rh, Ru, Ir, Pt, Pd, C, and N. An alloy having 65 weight % Fe and 35 weight % Mn (Fe-35Mn) has been reported by Hermawan et al. (“Fe—MN alloys for metallic biodegradable stents: Degradation and cell viability studies”, Acta Biomaterialia 6, pp. 1852-1860 (2010)), to have similar mechanical properties as 316 stainless, no indications of bio-toxicity, and corrosion rates similar to pure Fe.

In accordance with an embodiment of the invention, a corrosion activator species is added to the alloy in a low enough concentration to achieve a complete FCC austenite single phase during high temperature (about 1000 to 1200° C.) homogenization.

The alloy composition in accordance with embodiments of the invention may be described as Fe—X—Y, where the element X is an austenite stabilizing element selected from the group consisting of: Co, Ni, Mn, Cu, Re, Rh, Ru, Ir, Pt, Pd, C, and N, chosen alone or in combination with a high enough concentration to avoid the formation of ferromagnetic BCC phase during processing, and the element Y is a corrosion-activator species selected from the list consisting of: Au, and Pd, chosen alone or in combination with a low enough concentration to remain in solid FCC solution but high enough concentration to activate microscopically uniform corrosion to the desired rate. Table I lists example compositions (all numbers in weight %) in accordance with embodiments of the invention, which should not be considered to be limiting in any way.

TABLE I
Fe-based Alloys

| Example | Fe | Mn | Au | Pd | Pt | Ir |
|---|---|---|---|---|---|---|
| 1 | 64 | 35 | 1 | | | |
| 2 | 64.5 | 35 | | 0.5 | | |
| 3 | 70 | | | 30 | | |
| 4 | 63 | | 2 | | 35 | |
| 5 | 60 | | | 5 | 35 | |
| 6 | 63 | | 2 | | | 35 |
| 7 | 60 | | | 5 | | 35 |
| 8 | 54.8 | 45 | 0.18 | | | |
| 9 | 55.8 | 44 | 0.18 | | | |

In other words, alloys in accordance with embodiments of the present invention may include, but are not limited to, in wt %: Fe-35Mn-1Au, Fe-35Mn-0.5Pd, Fe-30Pd, Fe-35Pt-2Au, Fe-35Pt-5Pd, Fe-35Ir-2Au, Fe-35Ir-5Pd, Fe-45Mn-0.18Au, and Fe-44Mn-0.18Au, with the amount of Fe being the balance of the alloy.

Preliminary investigations into an example alloy of Fev34Mn-1Au (wt %) have indicated that the solubility limit is exceeded slightly, resulting second-phase particles even after homogenization at 1200° C. However, the matrix is still high in dissolved gold content after this treatment. Laboratory-scale corrosion tests indicate the corrosion behavior exhibits active corrosion in simulated body fluid.

An alloy according to Example 9 having a target composition of 55.8 wt % Fe, 44.0 wt % Mn, and 0.18 wt % Au was melted, extruded, swaged, and ground into two Sample rods, each rod having a diameter of about 0.190 inches (4.8 mm) and a length of about 37 inches (94 cm). The composition in the Sample was measured to include: 55.8 wt % Fe, 43.9 wt % Mn, 0.23 wt % Au, 35 ppm C, 230 ppm O, <10 ppm N, 68 ppm S, 51 ppm Mo, 2.4 ppm Si, 2.7 ppm Ni, 2.5 ppm Cu, and 1.2 ppm P, indicating that the Sample had relatively low impurity levels.

A microstructural analysis by scanning electron microscopy (SEM) was completed on the Sample and showed that there was no clear single-phase grain structure. Mn rich particles were detected and a minor Au signal was detected but not quantified. Corrosion pits in the vicinity of particles were also detected. The Sample was measured to have a Vickers hardness of 257 VHN, and predicted to have an ultimate tensile strength of 120 ksi, and similar mechanical properties to 316 stainless steel. The Sample was also subjected to X-ray diffraction. The results of the X-ray diffraction analysis suggested the presence of delta-ferrite (magnetic phase), as well as un-dissolved Au—Mn precipitates.

The thermodynamics of the Example 9 alloy were predicted. The 55.8 wt % Fe, 44.0 wt % Mn, and 0.18 wt % Au alloy is expected to be in the 2-phase region below about 900° C. Both Fe-rich FCC and MnAu-rich FCC phases are predicted. Re-homoginization at 1100° C. may be possible to re-dissolve the second phase particles, although the alloy may still be viable with second phase particles present.

The Example 9 alloy was homogenized, extruded, swaged, and re-homoginized. SEM analysis was used to estimate the phase fraction of the Re-homoginized Sample. The results indicated that about 1% of the area of the Re-homoginized Sample was second phase particles, although the number particles may have been over-estimated due to presence of corrosion pits. The microstructural analysis after homogenization indicated that there was significant grain growth and suggested successful dissolution of second phase particles that would otherwise pin grains to prevent growth. Small, evenly distributed corrosion spots were also observed. The Re-homoginized Sample was measured to have a Vickers hardness of 118 VHN, and predicted to have an ultimate tensile strength of 55 ksi, and similar mechanical properties to 316 stainless steel.

In accordance with an embodiment of the invention, the alloy may include less than 1% phase fraction of a second-phase particle that provides grain pinning to limit the grain growth during processing. Desirably, such compounds are non-metallic compounds that are not highly conductive to limit their effect on galvanic corrosion. Potential phases include, but are not limited to: NbC, TiC, VC, VN, etc.

While specific embodiments of the invention have been described above, it will be appreciated that the invention may be practiced otherwise than as described. The description above is intended to be illustrative, not limiting. For example, although the alloys are described as being used to make a stent, it should be appreciated that other medical devices may also be fabricated with such alloys in accordance with embodiments of the invention. Thus, it will be apparent to one skilled in the art that modifications may be made to the invention as described without departing from the scope of the claims set out below.

What is claimed is:

1. A stent comprising an iron-based alloy consisting essentially of: Fe—X—Y,
wherein X is at least one austenite stabilizing element selected from the group consisting of Co, Ni, Mn, Cu, Re, Rh, Ru, Ir, Pt, Pd, C, and N, and
wherein Y is at least one corrosion-activator species selected from the group consisting of Au, and Pd.

2. The stent according to claim 1, wherein the alloy consists essentially of:
64 weight % Fe, 35 weight % Mn, and 1 weight % Au.

3. The stent according to claim 1, wherein the alloy consists essentially of:
64.5 weight % Fe, 35 weight % Mn, and 0.5 weight % Pd.

4. The stent according to claim 1, wherein the alloy consists essentially of:
70 weight % Fe, and 30 weight % Pd.

5. The stent according to claim 1, wherein the alloy consists essentially of:
63 weight % Fe, 35 weight % Pt, and 2 weight % Au.

6. The stent according to claim 1, wherein the alloy consists essentially of:
60 weight % Fe, 35 weight % Pt, and 5 weight % Pd.

7. The stent according to claim 1, wherein the alloy consists essentially of:
63 weight % Fe, 35 weight % Ir, and 2 weight % Au.

8. The stent according to claim 1, wherein the alloy consists essentially of:
60 weight % Fe, 35 weight % Ir, and 5 weight % Pd.

9. The stent according to claim 1, wherein the alloy consists essentially of:
54.8 weight % Fe, 45 weight % Mn, and 0.18 weight % Au.

10. The stent according to claim 1, wherein the alloy consists essentially of:
55.8 weight % Fe, 44 weight % Mn, and 0.18 weight % Au.

11. The stent according to claim 1, further comprising less than 1% phase fraction of a second-phase particle configured to limit grain growth during processing of the alloy.

12. The stent according to claim 11, wherein the second-phase particle is selected from the group consisting of: NbC, TiC, VC, and VN.

13. The stent according to claim 1, wherein the stent further comprises a plurality of struts and a plurality of turns, wherein each turn connects a pair of adjacent struts.
